(19) United States
(12) Patent Application Publication
(10) Pub. No.: US 2005/0038331 A1
(43) Pub. Date: Feb. 17, 2005

(54) Insertable sensor assembly having a coupled inductor communicative system

(76) Inventors: Grayson Silaski, Portland, OR (US); Jonathan D. Birck, Portland, OR (US)

Correspondence Address: Timothy E. Siegel, Suite 206, 1868 Knapps Alley, West Linn, OR, 97068-4644, US

(21) Appl. No.: 10/640,978

(22) Filed: Aug. 14, 2003

(51) Int. Cl. A61B 5/05

(52) U.S. Cl. 600/347; 600/365

(57) ABSTRACT

A sensing system for determining the concentration of an analyte inside an animal body. The system includes an in vivo portion that is adapted to reside inside the animal body and that includes a sensing element that produces a sensing signal. In addition, a wearable ex vivo portion is physically attached to the in vivo portion. The ex vivo portion includes a first inductor that is adapted to receive a varying electro-magnetic signal and that has a pair of terminals. A variable load assembly presents a load across the pair of terminals and varies the load in response to the sensing signal. Also, an electronic monitoring unit is physically separate from the ex vivo portion and includes a second inductor, which is magnetically coupled to the first inductor and is adapted to transmit a varying electro-magnetic signal and to detect changes in load across the terminals of the first inductor.

### BACKGROUND OF THE INVENTION

Implanted medical devices frequently include a wire coil that is used to receive an electromagnetic wave broadcast from outside the body. Often the transmitted signal is used for information content and to power the embedded device. Referring to FIGS. 1 and 2, which show a system under development that includes a few prior art features that will be discussed here, a system 10 that is currently being developed for the sensing of glucose relies on a very thin “wire” type sensing element 12 that is inserted into the patient's subcutaneous tissue for a few days to a few weeks, depending on the particularities of the implementation. The system 10 also includes an ex vivo portion 14 that is physically attached to the sensing element 12, but resides outside the body and may be adhered to the skin.

Another portion, referred to as the electronics monitoring unit 16 or “EMU,” is in wireless communication with the ex vivo portion 14. The EMU 16 is typically quite a bit larger then the ex vivo portion 14 and may be worn by the patient by being suspended from his or her belt, or may be carried by the patient, for instance in a purse. The EMU 16 may be driven by standard AA batteries or by a rechargeable battery pack. It appears to be a commercial reality that smaller batteries have a higher cost per unit of stored energy than do larger batteries. The EMU 16 would typically be located a little less than a foot away from the ex vivo portion.

A stationary EMU 18 may be made available for nighttime use. The stationary EMU 18 would be approximately the size of a clock radio and would be made to plug into a standard wall electrical outlet. Accordingly, the cost of electric power is inconsequential in the use of EMU 18, which may, consequently be placed as much as about 4 meters (13 feet) away from the ex vivo portion 14.

One problem encountered in the design of the ex vivo portion is the reduction of ex vivo portion size and the amelioration of any hard areas or objects that must be located on the ex vivo portion. In addition, the cost of the ex vivo portion should be as low as possible so that it will be practical to dispose of the ex vivo portion after a few days use. To achieve these goals it is desirable to reduce battery size on the ex vivo portion as much as possible.

Although electromagnetic coupling has been used for communications between an ex vivo device and an implantable device, this has typically been for actuators that communicate with an ex vivo transceiver only occasionally, or for instances in which the ex vivo portion can be retained directly on the other side of the skin from the in vivo portion. For example, U.S. Pat. No. 6,015,386 describes a blood pressure measurement device having an implanted portion and an ex vivo portion that is strapped to the patient's wrist very near to the implanted portion. It appears that the movement of the device, caused by the pressure on the wall of a blood vessel, powers the implanted portion by causing changes in the inductance of an implanted inductor. This inductor is coupled to an inductor in the ex vivo portion, which detects the changes in inductance.

### SUMMARY

In a first separate aspect, the present invention is a sensing system for determining the concentration of an analyte inside an animal body. The system includes an in vivo portion that is adapted to reside inside the animal body and that includes a sensing element that produces a sensing signal. In addition, a wearable ex vivo portion is physically attached to the in vivo portion. The ex vivo portion includes a first inductor that is adapted to receive a varying electromagnetic signal and that has a pair of terminals. A variable load assembly presents a load across the pair of terminals and varies the load in response to the sensing signal. Also, an electronic monitoring unit is physically separate from the ex vivo portion and includes a second inductor, which is magnetically coupled to the first inductor and is adapted to transmit a varying electro-magnetic signal and to detect changes in load across the terminals of the first inductor.

In a second separate aspect, the present invention is an improvement to a biological sensing system that includes an in vivo portion, adapted to reside inside a patient, an ex vivo portion physically attached to the in vivo portion and having an antenna and an electronic monitoring unit that is physically separate from, but in wireless communication with, the ex vivo portion. The improvement is an electronic memory adapted to store memory contents in the ex vivo portion. Also, a transmitter in the electronic monitoring unit is adapted to transmit the memory signal to the ex vivo portion, directing the ex vivo portion to transmit the memory contents to the electronic monitoring unit.

The foregoing and other objectives, features and advantages of the invention will be more readily understood upon consideration of the following detailed description of the preferred embodiment(s), taken in conjunction with the accompanying drawings.

### BRIEF DESCRIPTION OF THE DRAWINGS

FIG. 1 is a block diagram representation of an analyte sensing assembly, according to the prior art.

FIG. 2 is a side view of an analyte sensing patch that is a part of the assembly of FIG. 1.

FIG. 3 is a simplified schematic diagram of the electronics of an analyte sensing patch according to the present invention.

### DETAILED DESCRIPTION OF THE PREFERRED EMBODIMENTS

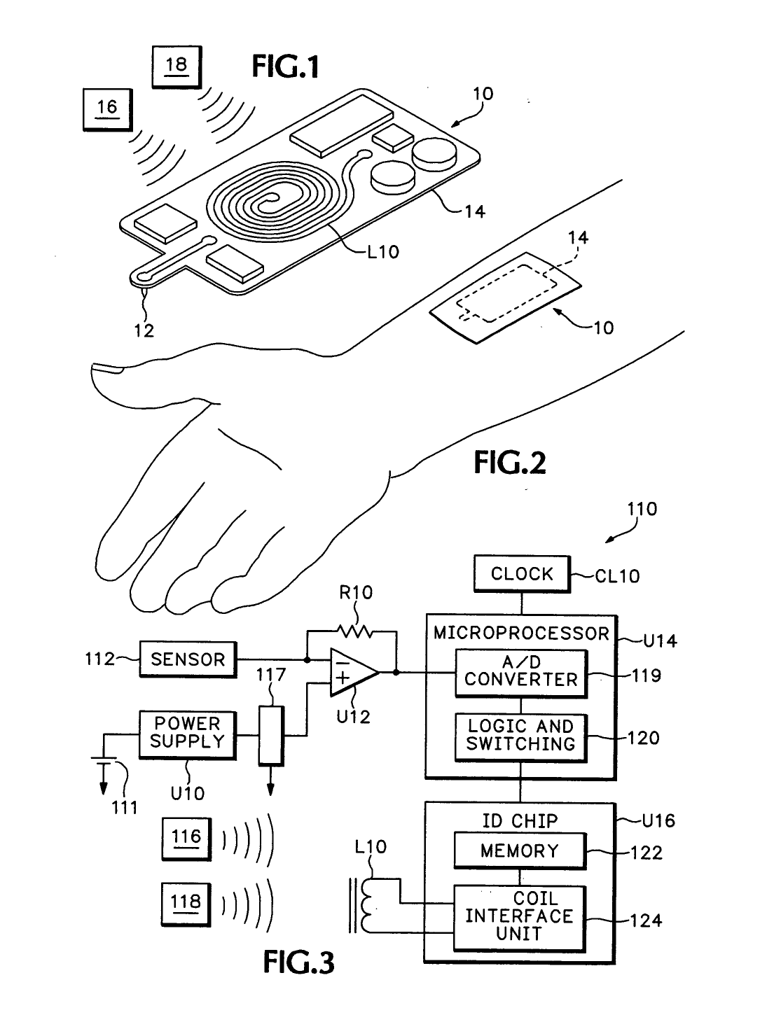

As described in the Background section, and as shown in FIGS. 1 and 2, in a preferred embodiment a glucose sensing and reporting assembly 10 includes an in vivo glucose sensing element 12 and an ex vivo portion 14, which transmits the data from sensing element 12 to an electronic monitoring unit 16. Together, elements 12 and 14 may be considered a glucose or analyte sensing patch.

In vivo portion 12 may be as described in U.S. Pat. No. 5,165,407. This portion must have a voltage placed across it of 0.65 VDC and produces a sensor current that is generally proportional to the concentration of glucose in body tissue. A typical value for the sensor current is about 5-10 nanoAmps.

In FIG. 3, elements that could be the same as an element in FIG. 1 or 2, are labeled with the same reference number as in FIG. 1 or 2, plus 100. Referring to FIG. 3, ex vivo portion includes a 3 VDC battery 111 that drives a power supply U10. The 2.048 VDC output of power supply U10 is fed into a voltage divider 117 having a 0.65 VDC output that drives the noninverting input of an Op Amp U12. The output of Op Amp U12 provides feedback to the inverting input of Op Amp U12, by way of precision 10 MOhm resistor R10. Consequently, the inverting input of Op Amp U12 tracks the noninverting input, which is fixed at a 0.65 VDC reference, thus biasing the sensing element 112. Accordingly, the output of Op Amp U12 equals 0.65 VDC+0.01*(sensing element current in nano Amps). If, for example, the sensing element current equals 5 nano Amps the output of U12 equals 0.70 VDC.

The op amp U12 output is fed into the input pin of microprocessor U14, which is preferably a Microchip model 16F676. Microprocessor U14 includes an internal analog-to-digital (A/D) converter 119, which converts the voltage at its input pin into a stream of 8 bit digital samples. A clock CL10 determines the rate at which microprocessor U14 operates. In one preferred embodiment, the clock CL10 is set at a rate of 32,768 Hz. The A/D converter 118, however, is operated only every 2 to 4 seconds when an internal counter of the microprocessor U14, which increments with every clock cycle, overflows. This activates an internal oscillator of the microprocessor U14, which then serves as a clock for the A/D, causing it to take one sample. A logic and switching unit 120 of microprocessor U14 averages or otherwise digital filters the sample into an updated glucose measurement value, before microprocessor U14 returns to an inactive state.

Approximately once per minute, during the brief active period of microprocessor U14, a finished glucose measurement word is sent in serial form into the input pin of an asset identification ID chip U16, which stores the word in a memory subunit 122. A coil interface unit 124, that is part of chip U16, effectively transmits the word in serial form over coil L10, by altering the resistance placed between the terminals of coil L10.

ID chip U16 may be an ATMEL AT24RF08C. This chip includes a dual port 256 word memory and may either send or receive over the pins that are connected with the coil. Chip U16 is controlled by the microprocessor U14 to store data coming from microprocessor U14, so that this data will not be lost if the EMU 116 (or a stationary EMU 118) fails to receive the data. Also, commands from the EMU 116 are stored in chip U16 and are sent into microprocessor U14.

The use of an asset identification ID chip U16 as part of ex vivo portion 14 provides a number of advantages over other methods of providing data to the EMU 116. This type of chip is typically used to tag items in a store in order to prevent theft or to tag an item that is part of a collection of items for easy identification. This type of chip has even been used to tag pets, so that a pet can be easily identified if lost or stolen.

A first advantage of using an asset ID chip is that chip U16 does not require energy from any other part of ex vivo portion 14 to effectively communicate with EMU 116. Rather chip U16 captures enough energy from coil L10 to operate internally, in order to effect the changes in resistance between the terminals of L10 that effectively transmit data to EMU 116.

Although chip U16 does require a small amount of energy to store data, the ability to store data greatly facilitates the EMU in saving energy, as a low EMU signal can generally be used for reading data from chip U16. The signal strength can be increased if it turns out that the EMU 116 did not provide enough energy to read the data from chip U16. Because the data is stored in chip U16, the data will still be available when the EMU 116 broadcasts with the requisite signal strength to read the data.

In the preferred embodiment, one glucose concentration measurement data word is created every minute. With this embodiment, using an ATMEL AT24RF08C for chip U16, 256 minutes (more than 4 hours) worth of data are stored. Accordingly, data could be stored and made available even in the event of an extended failure of EMU 116.

In one variant of this embodiment, microprocessor U14 is used to implement digital filtering of the data, so that the glucose concentration measurement word created every minute represents data collected over the entire minute interval. In another preferred embodiment, data words are formed more frequently than once per minute.

The Atmel AT24RF08C utilizes a 125 kHz frequency for communicating with the outside world. If this device were to be used for ID chip U16, then coil L10 would preferably be a ferrite-core inductor. If a different chip were to be used for asset ID chip U16, however, this could make possible the use of a higher frequency, making it more practical to implement coil L10 as an air core coil, as is shown in FIG. 1. In this embodiment the air core coil L10 is integrated into the flexible bandage structure that makes up the body of ex vivo portion 14 and is about two inches in diameter.

The terms and expressions that have been employed in the foregoing specification are used as terms of description and not of limitation. There is no intention, in the use of such terms and expressions, of excluding equivalents of the features shown and described or portions thereof, it being recognized that the scope of the invention is defined and limited only by the claims which follow.

1. A sensing system for determining the concentration of an analyte inside an animal body, said system including:
(a) an in vivo portion, adapted to reside inside said animal body and including a sensing element that produces a sensing signal;
(b) a wearable ex vivo portion physically attached to said in vivo portion, including:
(i) a first inductor adapted to receive a varying electromagnetic signal and having a pair of terminals; and
(ii) a variable load assembly presenting a load across said pair of terminals and varying said load in response to said sensing signal; and
(c) an electronic monitoring unit that is physically separate from said ex vivo portion and includes a second inductor, which is magnetically coupled to said first inductor and adapted to transmit a varying electromagnetic signal and to detect changes in load across said terminals of said first inductor.

2. The biological sensing system of claim 1, wherein said ex vivo portion also includes a battery.

3. The biological sensing system of claim 1, wherein said battery biases said sensing element.

4. The biological sensing system of claim 1, wherein said ex vivo portion also includes memory to store measurement values from said in vivo portion.

5. In a biological sensing system that includes an in vivo portion, adapted to reside inside a patient, an ex vivo portion physically attached to the in vivo portion and having an antenna, and an electronic monitoring unit that is physically separate from, but in wireless communication by way of electromagnetic signals with the ex vivo portion, an improvement comprising:
(a) electronic memory adapted to store memory contents in said ex vivo portion; and
(b) a transmitter in said electronic monitoring unit adapted to transmit a said electromagnetic signal to said ex vivo portion, directing said ex vivo portion to transmit at least a portion of said memory contents to said electronic monitoring unit.

6. The biological sensing system of claim 5 wherein said electronic monitoring unit includes a manual actuator adapted to permit a patient to enter a request for data and wherein said transmitter transmits a said electromagnetic signal requesting data to said ex vivo portion in response thereto.

7. The biological sensing system of claim 5 wherein said ex vivo portion is able to effectively transmit to said electronic monitoring unit by sequentially changing impedance across said antenna.
